(19) United States
(12) Patent Application Publication
(10) Pub. No.: US 2022/0370730 A1
(43) Pub. Date: Nov. 24, 2022

(54) DRUG INJECTION DEVICE

(71) Applicant: Tymphany worldwide Enterprises Limited, Grand Cayman (KY)

(72) Inventors: Yu-Cheng CHANG, Grand Cayman (KY); Tai-Shen CHEN, Grand Cayman (KY)

(21) Appl. No.: 17/749,559

(22) Filed: May 20, 2022

(30) Foreign Application Priority Data
May 20, 2021 (CN) 202110553667.4

(51) Int. Cl. A61M 5/32; A61M 5/34

(52) U.S. Cl. CPC A61M 5/3287; A61M 5/3293; A61M 5/343; A61M 5/344; A61M 2210/0612

(57) ABSTRACT

A drug injection device contains a needle holder, an ejector, a needle member, and an actuation assembly. The needle holder defines a drug delivery direction extending toward the injection end. The ejector contains a fixing segment that is fixed to the needle holder and located in the drug delivery direction, and the abutting segment extending toward the injection end and protruding beyond the needle holder. The needle member is movably arranged relative to the needle holder and contains a needle shaft connected to a needle hub coaxially arranged with the ejector for the abutting segment inserting into the needle shaft. The actuator assembly connects the needle holder and the needle shaft for driving the needle member between a first position and a second position with respect to the needle holder.

### CROSS REFERENCE TO RELATED APPLICATIONS

This application claims priority of Application No. 202110553667.4 filed in China on May 20, 2021 under 35 U.S.C. § 119, the entire contents of all of which are hereby incorporated by reference.

### TECHNICAL FIELD OF THE INVENTION

The present invention relates to drug injections, particularly to drug delivery injection devices which pierce human tissue for delivering drug to a predetermined location.

### BACKGROUND OF THE INVENTION

In addition to surgery, drug delivery can be also used to deal with eye diseases, especially for posterior segment eye diseases, such as age-related macular degeneration (AMD), diabetic retinopathy (DR), retinitis pigmentosa (RP), endophthalmitis, macular edema, etc., which makes it difficult to perform surgery. In addition, Glaucoma which is mainly caused by elevated intraocular pressure can also be treated by drug delivery.

As tissue barriers (e.g., corneal, conjunctival, blood-atrial water barrier, and blood-retinal barrier) limit drug delivery to the posterior region, a traditional approach is to pierce the eye with an instrument and deliver an effective dose of drug directly to the site location in the vitreous body. The traditional approaches for drug delivery in the vitreous include piercing the eye with a needle, using a jaw to enter the eye through the needle, releasing the jaw for drug delivery, and finally withdrawing the jaw. The microvascular, however, is prone to bleed during withdrawing the jaw from the eye, causing unanticipated sequelae.

### SUMMARY OF THE INVENTION

Aiming to solve the above issue, the present invention provides a drug injection device that solves the problem of drug delivered into human tissue, especially solves the problem of rupture of micro-vessels caused by delivering drug into the eyeball.

For the above and some related purposes, the present invention provides a drug injection device comprising a needle holder, an ejector, a needle member, and an actuation assembly. The needle holder defines a drug delivery direction. The drug delivery direction extends toward the injection end. The ejector comprises a fixing segment and an abutting segment. The fixing segment is fixed to the needle holder and disposed in the drug delivery direction. The abutting segment extends toward the injection end and protrudes beyond the needle holder. A needle member is movably disposed relative to the needle holder. The needle member comprises a needle hub and a needle shaft. The needle shaft is connected to the needle hub and the needle shaft is coaxially arranged with the ejector so that the abutting segment is inserted into the needle shaft. An actuation assembly connects the needle holder to the needle hub. The actuation assembly is used to drive the needle member relative to the needle holder between a first position and a second position, wherein the first position is close to the injection end and the second position is away from the injection end.

Preferably, the needle holder further comprises a concave space in which the fixing segment is fixed.

Preferably, the needle holder further comprises a rib section and a fixation post. The rib section is located in the concave space and is arranged parallel to the direction of drug delivery. The fixation post is located in the concave space with the drug delivery direction passing perpendicularly through the fixation post. The ejector further comprises a collar located at an end of the fixing segment away from the abutting segment, wherein the fixing segment is placed at the top edge of the rib section with the fixation post passing through the collar.

Preferably, the needle holder further comprises a guide rod located on the drug delivery direction and extending towards the injection end. The abutting segment passes through the guide rod along the axial direction of the guide rod and protrudes beyond the guide rod.

Preferably, the needle hub comprises a conical space for receiving the guide rod, and the needle shaft communicates with the conical space.

Preferably, the actuation assembly further comprises a linkage member, a sliding seat and a press member. The linkage member comprises an axle shaft, a rocker arm and a cam section. The rocker arm and the cam section extend radially along the axle shaft. The rocker arm further comprises a sliding slot hole. The axle shaft is disposed in the concave space and pivoted on the needle holder. The cam section is movable in an upper end and a lower end. The rocker arm is inclined towards the injection end when the cam section is in the upper end and the rocker arm is inclined in a direction away from the injection end when the cam section is at the lower end. A sliding seat is slidably disposed on the needle holder. The needle member is attached to the sliding seat. The sliding seat slides in a direction parallel to the drug delivery direction while the needle member moves between the first position and the second position. The sliding seat includes a sliding post, and the sliding seat is perpendicular to the drug delivery direction and penetrates the sliding slot hole of the rocker arm. A press member comprises a press block and a push rod. The press block comprises an upper surface and a lower surface. The lower surface faces the sliding seat. The push rod is disposed on the lower surface and abutting against the cam section to move the cam section from the upper end to the lower end.

Preferably, the rocker arm and the cam section maintain a spaced distance in the radial direction of the axle shaft. The fixing segment is located above the axle shaft, and the fixing segment passes between the rocker arm and the cam section.

Preferably, the cam section comprises a concave portion that is directed towards the front end of the push rod when the cam section is moved to a lower end.

Further, the drug injection device comprises a guiding cover being incorporated in the needle holder. The guiding cover comprises a guide block, and the sliding seat comprises a guide slot hole corresponding to the guide block. The guide slot hole extends in a direction parallel to the drug delivery direction. The guide block is slidably arranged in the guide slot hole.

Preferably, a top end of the guide block forms a stopper and the guide slot hole comprises a widened section. And, the width of the stopper is less than the width of the widened section and greater than the width of the rest of the guide slot hole.

Preferably, the guiding cover further comprises a first hook and a positioning post both extending towards the needle holder. The needle holder comprises a second hook and a positioning hole. The second hook is located in the concave space. The first hook and the second hook are hooked to each other, and the positioning post is inserted into the positioning hole.

Preferably, the sliding seat further comprises a body and a joining portion. The sliding post and the joining portion are connected to the body. The joining portion is located outside the needle holder corresponding to the guide rod with the needle hub fixed to the joining portion.

Preferably, the joining portion comprises a back plate, a clamping part and a positioning block. The back plate comprises a through hole used for the guide rod to pass through. The clamping part and the positioning block are provided on a side of the back plate away from the body. The needle hub comprises a flange. The clamping part is clamped on one edge of the flange, and the positioning block abuts against the other edge of the flange.

Further, the drug injection device comprises a housing containing a hollow space, wherein the housing has a side opening and a front opening. The needle holder, the linkage member, the fixing segment, the sliding seat and the needle hub are located in the hollow space, and the needle holder is fixed to the housing. The press block partially protrudes from the housing through the side opening, and the needle shaft and the abutting segment protrude from the housing through the front opening.

Preferably, the housing further comprises a hook fastener, and the needle holder comprises a fixing block. The hook fastener is hooked to the fixing block so as to fix the needle holder to the housing.

To summarize, the drug injection device proposed in the present invention is designed to smoothly pierce human tissue (such as eyeballs) with a needle shaft during drug delivery, and then the needle shaft is smoothly withdrawn. As there is no structure on the needle shaft and the ejector that hinders retraction, the drug injection device can effectively reduce the possibilities of microvascular rupturing and bleeding. In addition, the drug injection device of the present invention does not use pressure to push the drug against the resistance from human tissue, but makes the drug fall off naturally by withdrawing the needle shaft, thus avoiding the shaking caused by the force applied during the operation and further reducing the damage to human tissue. Therefore, the present invention effectively overcomes the problem encountered in the prior art that microvascular rupture caused by delivering drug into human tissues, especially the eyeball.

The above description is only an overview of the technical solution of the invention. To understand the technical means of the invention more clearly and to implement it in accordance with the contents of the specification, reference is made to the following detailed description of the invention with preferred embodiments and accompanied drawings.

### BRIEF DESCRIPTION OF THE DRAWINGS

Non-limiting embodiments of the invention will now be described, by way of example only, with reference to the accompanying drawings in which:

FIG. 1 is a three-dimensional exploded view of the drug injection device in an embodiment of the present invention;

FIG. 2 is a three-dimensional view of the drug injection device in an embodiment of the present invention;

FIG. 3 is a three-dimensional view of partial components of the drug injection device in an embodiment of the present invention;

FIG. 4 is a three-dimensional view of partial components of the drug injection device in an embodiment of the present invention;

FIG. 5 is a partial sectional view of partial components of the drug injection device in an embodiment of the present invention, showing a comparison before drug delivery and after drug delivery;

FIG. 6 is a side view of partial components of the drug injection device in an embodiment of the present invention, showing a comparison before drug delivery and after drug delivery;

FIG. 7 is a three-dimensional view of the press member and the linkage member in an embodiment of the present invention;

FIG. 8 is another three-dimensional view of the press member and the linkage member in an embodiment of the present invention;

FIG. 9 is a three-dimensional view of partial components of the drug injection device in an embodiment of the present invention;

FIG. 10 is a three-dimensional view of partial components of the drug injection device in an embodiment of the present invention;

FIG. 11 is a three-dimensional view of a sliding member in an embodiment of the present invention;

FIG. 12 is a three-dimensional view of the sliding member and the needle member in an embodiment of the present invention;

FIG. 13 is a three-dimensional exploded view of partial components of the drug injection device in an embodiment of the present invention;

FIG. 14 is a cross-sectional view of partial components of the drug injection device in an embodiment of the present invention, illustrating the state before drug delivery; and

FIG. 15 is a cross-sectional view of partial components of the drug injection device in an embodiment of the present invention, illustrating the state after drug delivery.

### DETAILED DESCRIPTION OF THE INVENTION

The embodiments of the present invention are described below by specific examples, and those skilled in the art can easily understand other advantages and effects of the present invention from the contents disclosed in this specification.

It should be noted that the embodiments in the present application and the features of the embodiments may be combined with each other without conflict. The present invention will be described in detail below with reference to the accompanying drawings and in conjunction with the embodiments. In order to enable those skilled in the art to better understand the solutions of the present invention, the technical solutions in the embodiments of the present invention will be clearly and completely described below with reference to the accompanying drawings in the embodiments of the present invention. Obviously, the described embodiments are only a part of the embodiments of the present invention, but not all embodiments. Based on the embodiments of the present invention, all other embodiments obtained by persons of ordinary skill in the art without creative efforts shall fall within the protection scope of the present invention.

It should be noted that the terms “first”, “second” and the like in the description and claims of the present invention and the above drawings are used to distinguish similar objects and need not to be used to describe a specific sequence or sequence. Furthermore, the terms “comprising” and “having”, and any variations thereof, are intended to cover non-exclusive inclusion. For example, a process, method, system, product or device comprising a series of steps or units is not necessarily limited to those expressly listed but may include other steps or units not expressly listed or inherent to these processes, methods, products or devices.

It should be noted that, unless otherwise expressly specified and limited, the terms “installed”, “connected” and “connected” should be understood in a broad sense. For example, it may be a fixed connection, a detachable connection, or an integral connection. It can be a mechanical connection or an electrical connection. It can be a direct connection, an indirect connection through an intermediate medium, or an internal connection between two components. For those of ordinary skill in the art, the specific meanings of the above terms in the present invention can be understood in specific situations.

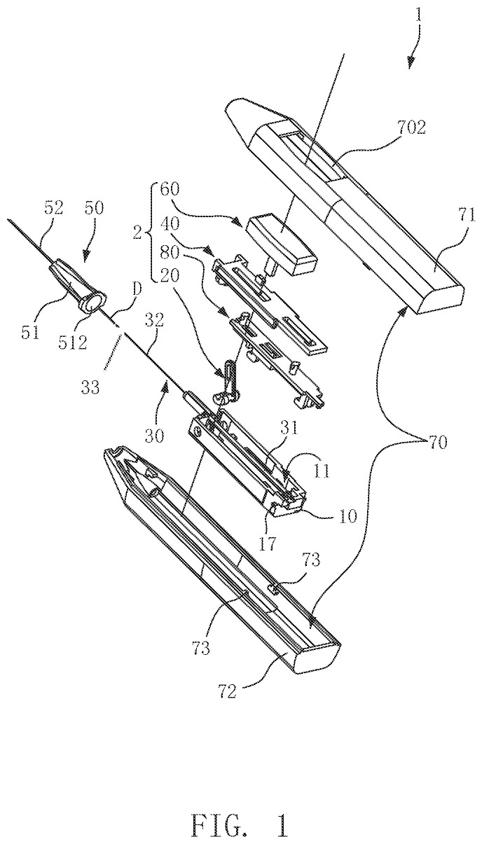

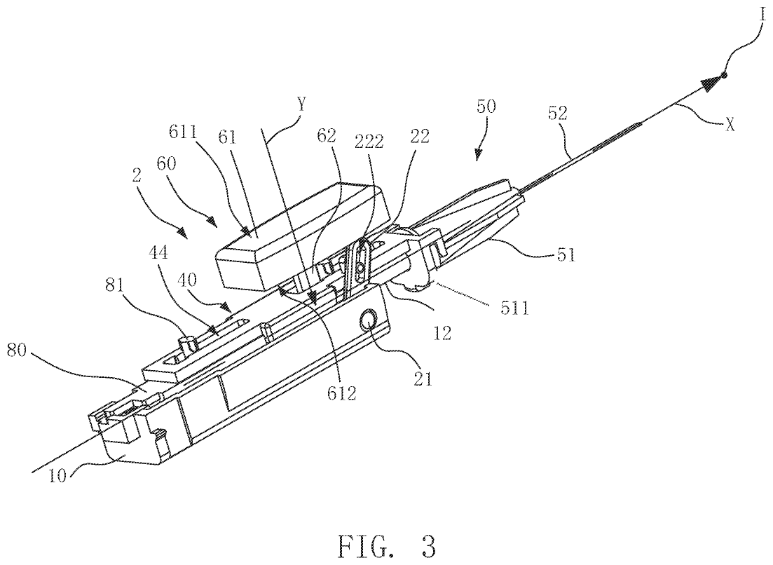

Referring to FIGS. 1 to 3, the embodiments of the present invention provide a drug injection device 1 comprising a needle holder 10, an ejector 30, a needle member 50, an actuation assembly 2, and a housing 70. The drug injection device 1 is used to deliver a drug D into human tissues using injection, such as injection into an eye to deliver the drug D, but the present invention does not exclude that drug injection device 1 can be used to perform delivery of the drug D into tissues in other parts of the human body.

As shown in FIGS. 1, 3 and 4, the needle holder 10 comprises a concave space 11. The needle holder 10 defines a drug delivery direction X and a pressing direction Y, where the drug delivery direction X extends toward the injection end I and the pressing direction Y that extends toward the concave space 11 intersects the drug delivery direction X. The needle holder 10 further comprises a guide rod 12 disposed in the drug delivery direction X and extending toward the injection end I.

As shown in FIGS. 1 and 4, the ejector 30 comprises a fixing segment 31 and an abutting segment 32, where the diameter of the fixing segment 31 is greater than the diameter of the abutting segment 32. The ejector 30 can be but not limited to an acupuncture needle. The fixing segment 31 is configured to hold the acupuncture needle, and the abutting segment 32 is configured for piercing the acupuncture needle. However, in the embodiment of the present invention, the front end of the abutting segment 32 may have a needle tip that does not provide the piercing function. The fixing segment 31 is fixed to the needle holder 10. Specifically, the fixing segment 31 is in the concave space 11 and in the direction X of drug delivery. The abutting segment 32 passes through the guide rod 12 along the axial direction of the guide rod 12. The abutting segment 32 extends toward the injection end I and protrudes beyond the needle holder 10 and the guide rod 12. In an embodiment of the present invention, an accommodating groove 122 is disposed on the guide rod 12, where the accommodating groove 122 is also located in the drug delivery direction X, extends toward the injection end I, and connects to the concave space 11. The abutting segment 32 is partially disposed in the accommodating groove 122 and passes through the guide rod 12. In different embodiments of the present invention, the accommodating groove 122 may be replaced by a through hole in the guide rod 12.

As shown in FIG. 4, the needle holder 10 preferably further comprises a rib section 13 and a fixation post 14. The rib section 13 is disposed in the concave space 11 and is arranged parallel to the drug delivery direction X. The fixation post 14 is disposed in the concave space 11 such that the drug delivery direction X passes vertically through the fixation post 14, and the rib 13 is disposed between the fixation post 14 and the guide rod 12. The ejector 30 further comprises a collar 33 disposed at an end of the fixing segment 31 away from the top abutting segment 32. The fixing segment 31 is disposed at the top edge of the rib 13 and is arranged parallel to the rib 13. The fixation post 14 passes through the collar 33 to fix the ejector 30 to the needle holder 10. Preferably, the portion of the fixing segment 31 that is attached to the abutting segment 32 can be fixed to the accommodating groove 122 by tightening or gluing.

As shown in FIGS. 1-3 and 5, the needle member 50 is movably disposed relative to the needle holder 10. The needle member 50 comprises a needle hub 51 and a needle shaft 52. The needle hub 51 comprises a conical space 512, where the guide rod 12 of the needle holder 10 is disposed in the conical space 512 for the needle hub 51 to cover the guide rod 12. The needle shaft 52 is attached to the needle hub 51 and is connected to the conical space 512. The needle shaft 52 and the ejector 30 are coaxially arranged, so that the abutting segment 32 is inserted into the needle shaft 52, and the needle shaft 52 is used to accommodate the drug D to be injected, such as the drug D for the treatment of glaucoma.

As shown in FIGS. 1-3 and 5, the actuation assembly 2 connects the needle holder 10 to the needle hub 51. The actuation assembly 2 is used for driving the needle member 50 to move between a first position P1 and a second position P2 relative to the needle holder 10, wherein the first position P1 is near the injection end I and said second position P2 is away from the injection end I. Specifically, the actuation assembly 2 of embodiments of the present invention comprises a linkage member 20, a sliding seat 40, and a press member 60.

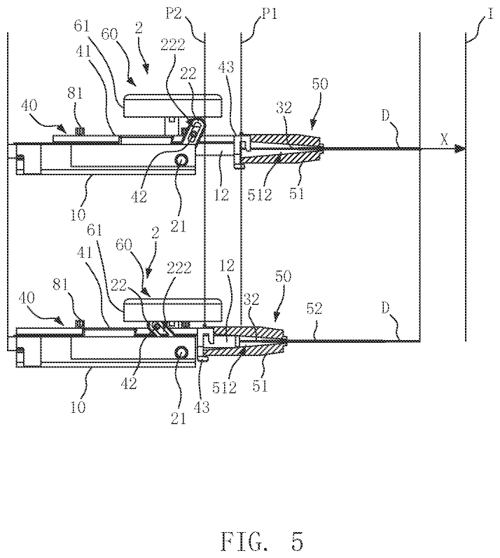

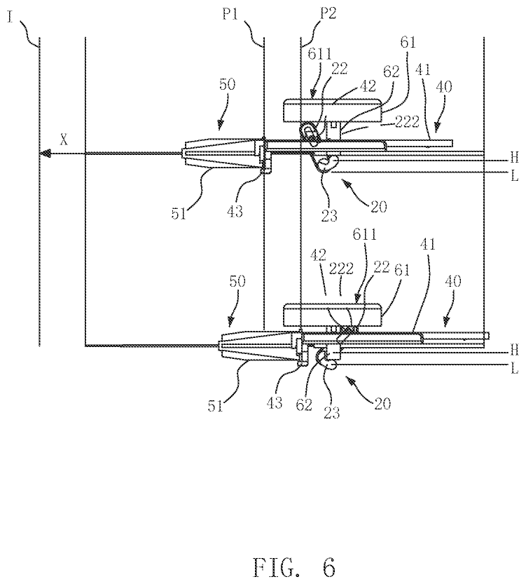

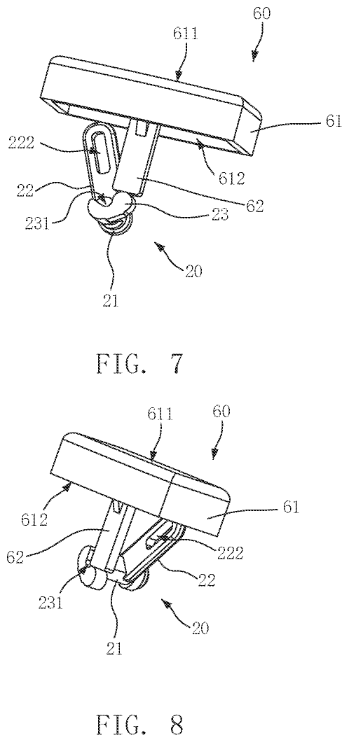

As shown in FIGS. 1 and 3-8, the linkage member 20 comprises an axle shaft 21, a rocker arm 22 and a cam section 23. The rocker arm 22 and the cam section 23 extend in a radial direction along the axle shaft 21. The rocker arm 22 also comprises a sliding slot hole 222 extending substantially in the direction of the extension of the rocker arm 22. The axle shaft 21 is disposed in the concave space 11 and is pivoted to the needle holder 10. The axial of axle shaft 21 is perpendicular to the drug delivery direction X, and the rocker arm 22 protrudes beyond the concave space 11. As shown in FIGS. 6 and 7, the cam section 23 is movable between the upper end H and the lower end L. When the cam section 23 is in the upper end H, the rocker arm 22 is tilted toward the injection end I. As shown in FIG. 6 and FIG. 8, when the cam section 23 is located at the lower end L, the rocker arm 22 is tilted toward the direction away from the injection end I. As shown in FIG. 4, the rocker arm 22 and the cam section 23 can be kept at a spacing distance in the radial direction of the axle shaft 21, wherein the fixing segment 31 of the ejector 30 is located above the axle shaft 21 in the concave space 11, and the fixing segment 31 passes between the rocker arm 22 and the cam section 23.

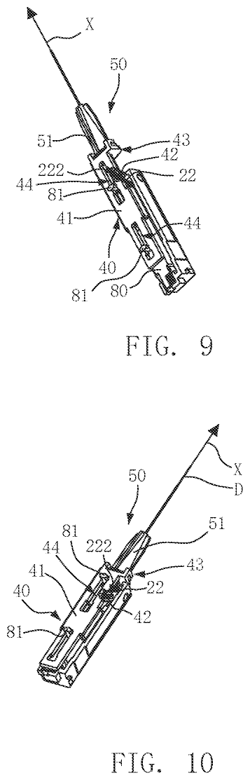

As shown in FIGS. 1, 3, 5, 6, 9 and 10, the sliding seat 40 is slidably disposed on the needle holder 10 and is arranged corresponding to the opening of the concave space 11. The needle member 50 is attached to the sliding seat 40 and the sliding direction of the sliding seat 40 is parallel to the drug delivery direction X. The sliding seat 40 can drive the needle member 50 moving between a first position P1 and a second position P2 with respect to the needle holder 10.

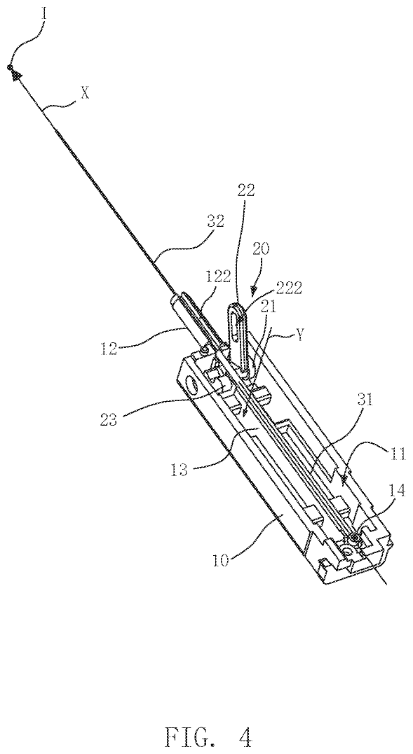

As in FIGS. 5, 6, 9, 10, 11 and 12, the sliding seat 40 comprises a body 41, a sliding post 42 and a joining portion 43. The sliding post 42 may be directly or indirectly attached to the body 41, protrude perpendicular to the drug delivery direction X, and penetrate the sliding slot hole 222 of the rocker arm 22. Specifically, the sliding seat 40 further comprises a lug 45 protruding from the upper surface 611 of the body 41 and disposed substantially at the edge of the body 41, and the sliding post 42 is disposed on the surface of the lug 45 and indirectly attached to the body 41. The joining portion 43 is attached to the body 41 and disposed outside the needle holder 10 corresponding to the guide rod 12. The needle hub 51 is fixed to the joining portion 43 such that the needle member 50 is linkable to the sliding seat 40.

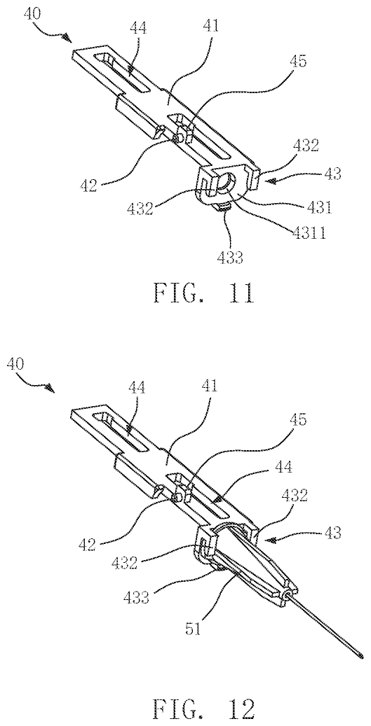

As shown in FIGS. 11 and 12, specifically, the joining portion 43 comprises a back plate 431, a clamping part 432, and a positioning block 433. The back plate 431 is arranged perpendicular to the body 41 and the back plate 431 comprises a through hole 4311. The through hole 4311 is for the guide rod 12 to pass through. The clamping part 432 and the positioning block 433 are disposed on the side of the back plate 431 away from the body 41. The needle hub 51 further comprises a flange 511. The clamping part 432 is clamped to one edge of the flange 511, and the positioning block 433 is held against the other edge of the flange 511 to fix the needle hub 51 to the joining portion 43 of the sliding seat 40 while the opening of the conical space 512 is kept facing the back plate 431.

As shown in FIGS. 5 and 6, when the rocker arm 22 is tilted toward the injection end I, the rocker arm 22 is linked to the sliding post 42 to position the needle member 50 in a first position P1. When the rocker arm 22 is tilted toward away from the injection end I, the rocker arm 22 is linked to the sliding post 42 to position the needle member 50 in a second position P2.

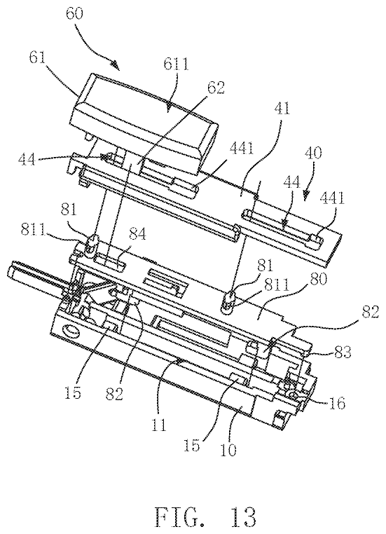

As shown in FIGS. 1, 3, 5, 9, 10 and 13, the actuation assembly 2 of the drug injection device 1 further comprises a guiding cover 80, which is combined with the needle holder 10 and at least partially covering the opening of the concave space 11. The guiding cover 80 comprises one or more guide blocks 81, and the sliding seat 40 comprises a guide slot hole 44 corresponding to the guide blocks 81. The extension direction of the guide slot hole 44 is parallel to the drug delivery direction X. The guide blocks 81 are slidably arranged in the guide slot hole 44, so that the sliding seat 40 is slidably disposed on the needle holder 10 through the guiding cover 80, and the sliding direction of the sliding seat 40 is restricted to be parallel to the drug delivery direction X. As shown in FIG. 13, the top end of the guide block 81 forms a stopper 811 and the guide slot hole 44 comprises a widened section 441, where the width of the stopper 811 is less than the width of the widened section 441 and greater than the width of the rest of the guide slot hole 44. Thus, during installation and removal of the sliding seat 40, the stopper 811 may be passed through the widened section 441 to install the guide block 81 into the guide slot hole 44. In the above process, the stopper 811 may be used to stop the sliding seat 40 to avoid the sliding seat 40 exiting the guiding cover 80.

As shown in FIG. 13, specifically, the guiding cover 80 further comprises one or plurality of first hooks 82 and a positioning post 83. The first hooks 82 and the positioning post 83 extend toward the needle holder 10. The needle holder 10 comprises a second hook 15 and a positioning hole 16. The second hook 15 is disposed in the concave space 11 and the positioning hole 16 is oriented towards the guiding cover 80. The first hooks 82 and the second hook 15 are hooked to each other and the positioning post 83 is embedded in the positioning hole 16 for securing the guiding cover 80 to the needle holder 10.

As shown in FIGS. 1, 3, 7, 8 and 13, the press member 60 comprises a press block 61 and a push rod 62. The press block 61 comprises an upper surface 611 and a lower surface 612 that faces the sliding seat 40. Also referring to FIG. 6, the push rod 62 is provided on the lower surface 612, passes through the sliding seat 40 and the guiding cover 80, and rests against the cam section 23 of the linkage 20 to push the cam section 23 from the upper end H to the lower end L. The push rod 62 extends substantially in the pressing direction Y. The push rod 62 extends from the upper end H to the lower end L. The push rod 62 extends substantially in the pressing direction Y. As shown in FIG. 13, the sliding seat 40 and the guiding cover 80 may each be arranged with a through hole 84 for the push rod 62 to pass through, and a partial of the guide slot hole 44 may be directly utilized as a through hole in the sliding seat 40. Alternatively, the sliding seat 40 and the guiding cover 80 are structurally designed to comprise separate notches to avoid the necessary space for the push rod 62 to pass through.

As shown in FIG. 6, the upper surface 611 of the press block 61 is used for pressing, so that the press block 61 moves toward the linkage 20 in the pressing direction Y and drives the front end of the push rod 62 against the cam section 23. The push rod 62 pushes the cam section 23 so that the cam section 23 moves from the upper end H to the lower end L, thereby changing the tilt direction of the rocker arm 22. As shown in FIGS. 7 and 8, the cam section 23 further comprises a concave portion 231. When the cam section 23 moves to the lower end L, the concave portion 231 faces the front end of the push rod 62 to capture the front end of the push rod 62 and prevent the push rod 62 from slipping off the sides of the cam section 23.

As shown in FIGS. 1, 2, 14 and 15, the interior of the housing 70 comprises a hollow space 701, and the housing 70 comprises a side opening 702, a front opening 703 and a hollow space 701 disposed in the interior of the housing 70. The needle holder 10, linkage 20, fixing segment 31, sliding seat 40 and needle hub 51 are located in the hollow space 701, and the needle holder 10 is fixed to the housing 70. The press block 61 of the press member 60 passes through the side opening 702 so that the press block 61 partially protrudes from the housing 70 and the upper surface 611 is exposed outside the housing 70. In addition, the contour of the side opening 702 substantially matches the press block 61 to limit linear movement of the press block 61 along the pressing direction Y and to ensure that the front end of the push rod 62 can be aligned with the cam section 23. The needle shaft 52 and the abutting segment 31 protrude from the housing 70 through the front opening 703.

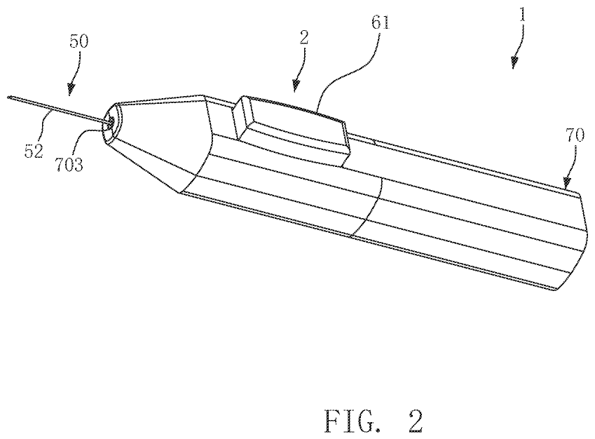

As shown in FIG. 1 and FIG. 2, specifically, the housing 70 comprises an upper housing 71 connected to a lower housing 72. The side opening 702 is located in the upper housing 71, and the front opening 703 is located in the butt joints of the upper housing 71 and the lower housing 72. The housing 70 further comprises hook fasteners 73, which are respectively disposed on the upper housing 71 and the lower housing 72. The needle holder 10 comprises a fixing block 17, where the hook fasteners 73 can be hooked to the fixing block 17 to fix the needle holder 10 to the housing 70.

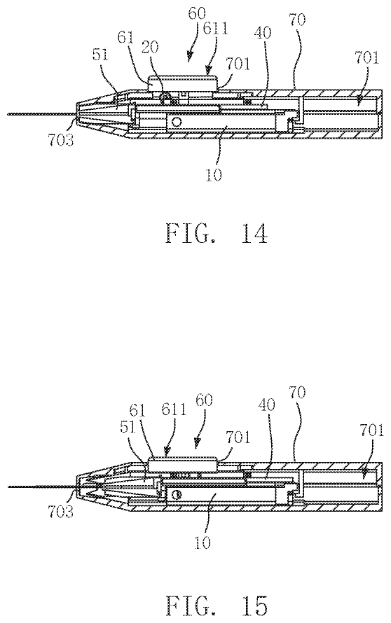

As shown in FIG. 5, FIG. 6 and FIG. 14, prior to performing the injection of the drug D, the abutting segment 31 of the ejector 30 is partially disposed in the needle shaft 52 such that a segment of the needle shaft 52 still has space near its tip to be pre-filled with the drug D to be delivered. At the same time, the guide rod 12 of the needle holder 10 may be pointing toward the conical space 511 of the needle hub 21 but still disposed outside the conical space 511. The needle shaft 52 is used to pierce the human body such that the tip of the needle shaft 52 is disposed at a predetermined location in the human tissue. For example, the needle shaft 52 pierces the eyeball such that the tip of the needle shaft 52 reaches the vitreous body behind the lens. At the same time, the cam section 23 is disposed in the upper end H. The rocker arm 22 is angled toward the injection end I. The slide seat 40 is disposed in the first position P1, and the press block 61 is unpressed and protrudes from the housing 70.

As shown in FIGS. 5, 6, and 15, the drug injection device 1 can be operated by a medical person in a hand-held position, or it can be mounted on a robotic arm and operated with the aid of a robotic arm, or the actuation assembly itself has some other form of power source to drive the sliding seat 40. Next, while the position of the drug injection device 1 is fixed (without moving the housing 70), the press block 61 of the press member 60 is pressed with a finger or a mechanical arm, wherein the press block 61 moves the push the rod 62 and pushes the cam section 23 to the lower end L. Thus, the rocker arm 22 is driven and tilted in the direction away from the injection end I. Thus, the rocker arm 22, the push rod 62, and the sliding seat 40 are driven to the lower end L. As a result, the rocker arm 22, the sliding post 42 and the sliding seat 40 are linked to drive the needle member 50 to move to the second position P2 relative to the needle holder 10. During this process, the housing 70, the needle holder 10, the guiding cover 80, and the ejector 30 are maintained to be stationary. During this process, the guide rod 12 can be guided by the wall of the conical space 511 to correct the offset of the ejector 30 and ensure that the needle shaft 52 and the ejector 30 are in a coaxial configuration, so that the abutting segment 32 is inserted into the needle shaft 52 without bending. Thus, the guide rod 12 can reduce the length of the overhang of the abutting segment 32 and avoid the abutting segment 32 from wobbling.

The needle member 50 is driven by the sliding seat 40 to move in the opposite direction of the drug delivery direction X. Thus, the abutting segment 31 is closer to or even protrudes from the tip of the needle shaft 52, which allows the drug D to be delivered to disengage from the needle shaft 52 and enter the intended delivery position. During the above process, the positions of the drug D and ejector are remained to be stationary (give that the housing 70 is not moving), but instead the needle shaft 52 is withdrawn in order to deliver and dislodge the drug D.

Ultimately, the drug injection device 1 of the present invention first penetrates the human tissues with the needle shaft 52, and then withdraws the needle shaft 52 with a small stroke to disengage the drug D. Instead of pushing the drug D by moving the ejector 30 toward the front of the needle shaft 52, there is no need to push the drug D by advancing the ejector 30 or the syringe core to counter the resistance of the human tissues during the drug delivery process, so the drug injection device 1 of the present invention can reduce the shaking caused by the force applied during the operation, and further reduce the damage to the human tissues.

In summary, in the process of injecting a drug, the drug injection device 1 proposed in the present invention smoothly penetrates human tissues (such as an eye) with a needle shaft and then smoothly withdraws the needle shaft. As there is no structure on the needle shaft 51 and the ejector 30 that prevents retraction, the states of microvascular rupture and bleeding can be effectively reduced. In addition, the drug injection device 1 of the present invention does not use pressure against the human tissues resistance to push the drug D, but withdraws the needle shaft 51 to make the drug D fall off naturally, so as to avoid the shaking caused by force during the operation. Further reduce damage to human tissues. Therefore, the present invention can effectively overcome the problem of the microvascular rupture caused by injecting the drug D to the human tissues (especially, the injecting of the drug D to the eyeball) in the prior art.

The drug injection device 1 provided by the embodiments of the present invention has been introduced in detail above. For those skilled in the art, some modifications may be made based on the ideas of the embodiment of the present invention. However, the content of this specification should not be construed as a limitation to the present invention, and all equivalent modifications or changes made according to the spirit and technical idea of the present invention should still be covered by the claims of the present invention.

1. A drug injection device, comprising:
a needle holder defining a drug delivery direction, wherein said drug delivery direction extends towards an injection end;
an ejector comprising:
a fixing segment fixed to said needle holder and arranged along said drug delivery direction; and
an abutting segment extending towards the injection end and protruding beyond the needle holder;
a needle member, movably arranged relative to said needle holder, said needle member comprising:
a needle hub; and
a needle shaft attached to said needle hub, said needle shaft being coaxially arranged with said ejector such that said abutting segment is inserted into said needle shaft; and
an actuation assembly connected to said needle holder and said needle hub,
wherein said actuation assembly is configured to drive said needle member to move between a first position and a second position relative to said needle holder, and
wherein said first position is proximal to said injection end and said second position is distal from said injection end.

2. The drug injection device according to claim 1, wherein said needle holder further comprises a concave space, and said fixing segment is located in said concave space.

3. The drug injection device according to claim 2, wherein said needle holder further comprises:
a rib section disposed in said concave space and is arranged parallel to said drug delivery direction; and
a fixation post located in said concave space with said drug delivery direction perpendicularly passing through said fixation post
wherein said ejector further comprises a collar, located at an end of said fixing segment away from said abutting segment, and
wherein said fixing segment is placed at the top edge of said rib section with said fixation post passing through said collar.

4. The drug injection device according to claim 2, wherein said needle holder further comprises a guide rod located on said drug delivery direction and extending towards said injection end, and
wherein said abutting segment passes through said guide rod along an axial direction of said guide rod and protrudes beyond said guide rod.

5. The drug injection device according to claim 4, wherein said needle hub comprises a conical space for receiving said guide rod, and said needle shaft communicates with said conical space.

6. The drug injection device according to claim 4, wherein said actuation assembly further comprises:
a linkage member comprising:
an axle shaft;
a rocker arm; and
a cam section,
wherein said rocker arm and said cam section extend along the radical direction of said axle shaft,
wherein said rocker arm comprises a sliding slot hole,
wherein said axle shaft is disposed in said concave space and pivoted to said needle holder,
wherein said cam section is movable at an upper end and a lower end, and
wherein said rocker arm is inclined towards said injection end when said cam section is at said upper end, and said rocker arm is inclined in a direction away from said injection end when said cam section is at said lower end;
a sliding seat disposed slidably on said needle holder, wherein said needle member is attached to said sliding said, wherein said sliding seat slides in a direction parallel to said drug delivery direction while said needle member moves between said first position and said second position, wherein said sliding seat includes a sliding post, and wherein said sliding post is perpendicular to said drug delivery direction and penetrates said sliding slot hole of said rocker arm; and
a press member comprising:
a press block comprising:
an upper surface; and
a lower surface facing said sliding seat; and
a push rod disposed on said lower surface and abutting against said cam section to push said cam section from said upper end to said lower end.

7. The drug injection device according to claim 6, wherein said rocker arm and said cam section maintain a spaced distance in the radial direction of said axle shaft,
wherein said fixing segment is located above said axle shaft, and
wherein said fixing segment passes between said rocker arm and said cam section.

8. The drug injection device according to claim 6, wherein said cam section comprises a concave portion directed towards the front end of said push rod when said cam section is moved to said lower end.

9. The drug injection device according to claim 6, further comprising a guiding cover being incorporated in said needle holder, said guiding cover comprising a guide block,
wherein said sliding seat comprises a guide slot hole corresponding to said guide block, and
wherein said guide slot hole extends in a direction parallel to said drug delivery direction, and said guide block is slidably arranged in said guide slot hole.

10. The drug injection device according to claim 9, wherein a top end of said guide block forms a stopper,
wherein said guide slot hole comprises a widened section, and
wherein a width of said stopper is less than a width of said widened section and greater than a width of a rest of said guide slot hole.

11. The drug injection device according to claim 9, wherein said guiding cover further comprises:
a first hook extending towards said needle holder; and
a positioning post extending towards said needle holder, and
wherein said needle holder comprises:
a second hook located in said concave space; and
a positioning hole, and
wherein said first hook and said second hook are hooked to each other, and said positioning post is inserted into said positioning hole.

12. The drug injection device according to claim 6, wherein said sliding seat further comprises:
a body connected to said body; and
a joining portion connected to said body, said joining portion being located outside said needle holder corresponding to said guide rod, and said needle hub is fixed to said joining portion.

13. The drug injection device according to claim 12, wherein said joining portion comprises:
a back plate comprising a through hole used for said guide rod to pass through;
a clamping part provided on a side of said back plate away from said body; and
a positioning block provided on a side of said back plate away from said body,
wherein said needle hub comprises a flange, said clamping part is clamped on one edge of said flange, and said positioning block abuts against the other edge of said flange.

14. The drug injection device according to claim 6, further comprising a housing containing a hollow space,
wherein said housing has a side opening and a front opening,
wherein said needle holder, said linkage member, said fixing segment, said sliding seat and said needle hub are located in said hollow space, and said needle holder is fixed to said housing, and
wherein said press block protrudes partially from said housing through said side opening, and said needle shaft and said abutting segment protrude from said housing through said front opening.

15. The drug injection device according to claim 14, wherein said housing further comprises a hook fastener,
wherein said needle holder comprises a fixing block, and
wherein said hook fastener is hooked to said fixing block so as to fix said needle holder to said housing.
